(19) United States
(12) Patent Application Publication
(10) Pub. No.: US 2025/0025068 A1
(43) Pub. Date: Jan. 23, 2025

(54) METHOD OF PROVIDING HEALTH CARE SERVICE, SERVER AND HEALTH CARE DEVICE

(71) Applicant: SAMSUNG ELECTRONICS CO., LTD., Suwon-si (KR)

(72) Inventor: Seonggyu KIM, Suwon-si (KR)

(73) Assignee: SAMSUNG ELECTRONICS CO., LTD., Suwon-si (KR)

(21) Appl. No.: 18/667,808

(22) Filed: May 17, 2024

Related U.S. Application Data
(63) Continuation of application No. PCT/KR2024/006228, filed on May 9, 2024.

(30) Foreign Application Priority Data
Jul. 19, 2023 (KR) 10-2023-0094186

(51) Int. Cl. A61B 5/117; A61B 5/0205; G16H 20/30

(52) U.S. Cl. CPC A61B 5/117; A61B 5/0205; G16H 20/30

(57) ABSTRACT

A method of providing a health care service, including: collecting, by a health care device, biometric information associated with a user corresponding to the health care device, through at least one sensor; identifying the user based on the biometric information and a user database stored in a server; and performing, by the health care device, a personalized care operation corresponding to the identified user.

### CROSS-REFERENCE TO RELATED APPLICATIONS

This application is a continuation of International Application No. PCT/KR2024/006228, filed on May 9, 2024, in the Korean Intellectual Property Receiving Office, which is based on and claims priority to Korean Patent Application Number 10-2023-0094186 filed on Jul. 19, 2023, in the Korean Intellectual Property Office, the disclosures of which are incorporated by reference herein in their entireties.

### BACKGROUND

### 1. Field

The disclosure relates to a method of providing a health care service, server and health care device, and more particularly, to a method of providing a health care service, server and health care device for caring for health of a user.

### 2. Description of Related Art

Health care devices may be used to manage health and disease in a home health care environment. For example, such health care devices may include smart beds that improve quality of sleep, massage chairs and bed-type massage devices that relax tight muscles and help relief of fatigue through massage, and treadmills, indoor bikes, etc., for indoor exercise.

However, these health care devices may not provide health care services optimized for the actual condition of a user. For example, these health care devices may operate i by selecting several modes defined in advance. However, users may have various demands depending on their respective preferences and conditions. With the selection of largely categorized modes, it may be difficult to provide a health care service optimized for the actual condition of a particular user. Users may desire to control the health care device according to their individual demands, but this may not be satisfied by the some mode selection methods. This may prevent the users from achieving a desired effect sufficiently through the health care device.

Furthermore, each user may desire a customized health care service. However, it may be difficult to select and control modes and settings individually, and the user may also need to spend time and effort to find a combination according to the preference of the user. The user may also experience discomfort at having to operate the health device each time to choose a desired combination.

### SUMMARY

Provided is a method of providing a health care service, server and health care device capable of automatically providing a health care service personalized for each user.

Technological objectives of the disclosure are not limited to what are mentioned above, and throughout the specification, it will be clearly appreciated by those of ordinary skill in the art that there may be other technological objectives unmentioned.

Additional aspects will be set forth in part in the description which follows and, in part, will be apparent from the description, or may be learned by practice of the presented embodiments.

In accordance with an aspect of the disclosure, a method of providing a health care service includes: collecting, by a health care device, biometric information associated with a user corresponding to the health care device, through at least one sensor; identifying the user based on the biometric information and a user database stored in a server; and performing, by the health care device, a personalized care operation corresponding to the identified user.

The identifying the user may include identifying the user by comparing the biometric information with registered biometric information stored in the user database.

The performing the personalized care operation may include operating according to a mode that is preferred by the identified user from among a plurality of control modes of the health care device.

The method may further include determining a change in a condition of the identified user by comparing a current value of the biometric information with a previous value of the biometric information, and the performing the personalized care operation may include performing the personalized care operation based on the change in the condition of the identified user.

The biometric information may include information about at least one of a height, a weight, a blood pressure, a heart rate, a heart rate variability, an oxygen saturation, a respiratory rate, a body temperature, fingerprints, an iris, a face, a vein, a voice, or a snoring sound.

The previous value may include an average value of the biometric information obtained over a predetermined period of time.

The performing the personalized care operation may include displaying information about at least one of a recommended mode, a medical treatment, and a health care method to the identified user based on the change in the condition of the identified user.

The method may further include determining a customized mode for the health care device based on the change in the condition of the identified user, and the performing the personalized care operation may include operating the health care device according to the customized mode.

The customized mode may include at least one mode from among a plurality of control modes of the health care device, or at least one modified mode having one or more changed mode variables from among the plurality of control modes.

Based on the health care device including a smart bed, the customized mode may include at least one control mode corresponding to the change in the condition of the identified user from among a plurality of control modes of the smart bed, or at least one modified mode from among the plurality of control modes in which one or more of a bed posture, a mattress hardness, a heat, a humidity and an illumination is changed.

Based on the health care device including a smart massage device, the customized mode may include at least one control mode corresponding to the change in the condition of the identified user from among a plurality of control modes of the smart massage device, or at least one modified mode from among the plurality of control modes in which one or more of a massage type, a massage portion, a massage intensity, a heating portion, and a heating intensity is changed.

Based on the health care device including smart workout equipment, the customized mode may include at least one control mode corresponding to the change in the condition of the identified user from among a plurality of control modes of the smart workout equipment, or at least one modified mode from among the plurality of control modes in which one or more of a workout type and a workout intensity is changed.

In accordance with an aspect of the disclosure, a server includes: a communicator; and a controller configured to: receive biometric information associated with a user who uses a health care device through the communicator, identify the user based on the biometric information, and transmit, to the health care device, a control command instructing the health care device to perform a personalized care operation corresponding to the identified user.

The controller may be further configured to transmit, to the health care device, a control command instructing the health care device to operate according to a mode that is preferred by the identified user from among a plurality of control modes of the health care device.

The controller may be further configured to determine a change in a condition of the identified user by comparing a current value of the biometric information with a previous value of the biometric information, and transmit, to the health care device, a control command instructing the health care device to perform a personalized care operation corresponding to the change in the condition of the identified user.

According to the disclosure, a method of providing a health care service, server and health care device is capable of automatically providing a health care service personalized for each user.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 illustrates a network system including a health care device and a server, according to an embodiment.

FIG. 2 illustrates an example of a control block of a server, according to an embodiment.

FIG. 3 is a flowchart for describing a process for providing a health care service,

according to an embodiment.

FIG. 4 is a flowchart for describing a process for performing a mode preferred by an identified user in a method of providing a health care service, according to an embodiment.

FIG. 5 is a flowchart for describing a process for performing a customized mode corresponding to a change in condition of an identified user in a method of providing a health care service, according to an embodiment.

FIG. 6 illustrates a user database stored in a server, according to an embodiment.

FIG. 7 illustrates a sleep state of each user in a user database stored in a server, according to an embodiment.

FIG. 8 illustrates a process for outputting information about a recommended mode, medical treatment or a health care method corresponding to a change in user condition in a method of providing a health care service, according to an embodiment.

FIG. 9 illustrates an example of a control block of a health care device, according to an embodiment.

FIG. 10 illustrates an occasion when a health care device is a smart bed, according to an embodiment.

FIG. 11 illustrates an occasion when a health care device is a smart massage device, according to an embodiment.

FIG. 12 illustrates an occasion when a health care device is a smart workout equipment, according to an embodiment.

FIG. 13 illustrates an example of a control block of a health care device, according to another embodiment.

### DETAILED DESCRIPTION

Embodiments and features as described and illustrated in the disclosure are merely examples, and there may be various modifications replacing the embodiments and drawings at the time of filing this application.

The terminology used herein is for the purpose of describing particular embodiments only, and is not intended to limit the disclosure.

For example, the singular forms “a”, “an” and “the” as herein used are intended to include the plural forms as well, unless the context clearly indicates otherwise.

Throughout the description, “A or B”, “at least one of A and B”, “at least one of A or B”, “A, B or C”, “at least one of A, B and C”, and “at least one of A, B, or C” may each include any one or all the possible combinations of A, B and C. For example, the “at least one of A or B” may include “A”, “B”, or “A and B”. The “at least one of A and B” may include “A”, “B”, or “A and B”. In another example, the “at least one of A, B or C” may include “A”, “B”, C”, “A and B”, “B and C”, “A and C” or “A, B and C”. The “at least one of A, B and C” may include “A”, “B”, C”, “A and B”, “B and C”, “A and C” or “A, B and C”.

Throughout the specification, the expression “and/or” may be interpreted to include a combination or any of associated elements. For example, an expression “A, B and/or C” may include “A”, “B”, C”, “A and B”, “B and C”, “A and C” or “A, B and C”.

Terms like “first”, “second”, etc., may be simply used to distinguish an element from another, without limiting the elements in a certain sense (e.g., in terms of importance or order).

When an element is mentioned as being “coupled” or “connected” to another element with or without an adverb “functionally” or “operatively”, this means that the element may be connected to the other element directly (e.g., wiredly), wirelessly, or through a third element.

It will be further understood that the terms “comprise” and/or “comprising,” when used in this disclosure, specify the presence of stated features, integers, steps, operations, elements, parts or combinations thereof, but do not preclude the possible presence or addition of one or more other features, integers, steps, operations, elements, components, and/or groups thereof.

When an element is mentioned as being “connected to”, “coupled to”, “supported on” or “contacting” another element, this includes a case in which the elements are directly connected to, coupled to, supported on or contact each other, and also a case in which the elements are connected to, coupled to, supported on or contact each other through a third element.

Throughout the disclosure, when an element is described as being located “on” another element, this means that the element may abut on the other element, and also that a third element may exist between the two elements.

Furthermore, the terms, such as “˜part”, “˜block”, “˜member”, “˜module”, etc., may refer to a unit of handling at least one function or operation. For example, the terms may refer to at least one process handled by hardware such as a field-programmable gate array (FPGA)/application specific integrated circuit (ASIC), etc., software stored in a memory, or at least one processor.

Reference numerals used for method steps are only used to identify the respective steps, and not to limit an order of the steps. Thus, unless the context clearly indicates otherwise, the written order may also be practiced otherwise.

Reference will now be made in detail to embodiments of the disclosure, examples of which are illustrated in the accompanying drawings.

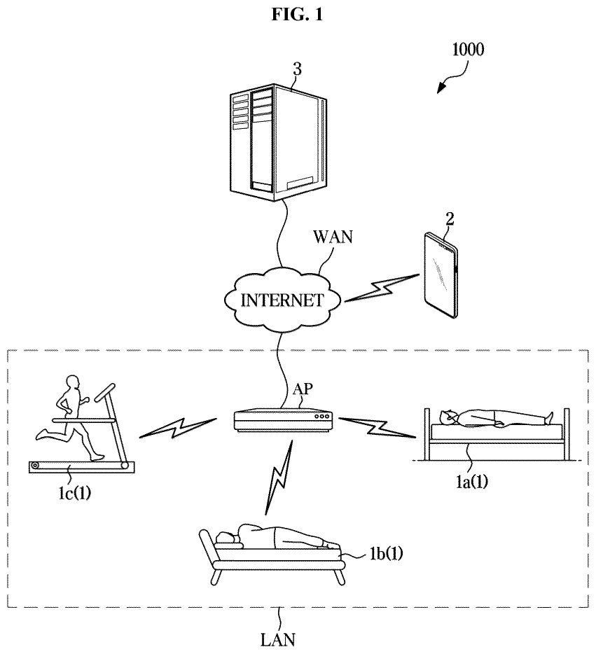

FIG. 1 illustrates a network system including a health care device, according to an embodiment.

Referring to FIG. 1, the network system 1000 according to an embodiment may include a health care device 1, a user equipment 2 and/or a server 3.

The health care device 1 may include a communication module for communicating with the user equipment 2, the server 3 and/or another health care device, a user interface for receiving user inputs or outputting information to the user, at least one processor for controlling operations of the health care device 1, and at least one memory for storing a program for controlling the operations of the health care device 1.

The health care device 1 may be, or may include, a device for managing user's health, disease, etc. For example, the health care device 1 may include at least one of a smart bed 1a, a smart massage device 1b and a smart workout equipment 1c. The smart bed 1a may include a smart mattress. The smart massage device 1b may include a massage chair, a bed-type massage device, etc. The smart workout equipment 1c may include a treadmill, an indoor bike, etc.

The server 3 may include a communication module for communicating with the health care device 1, another server and/or the user equipment 2. The server 3 may include at least one processor for processing data received from the other server and/or the user equipment 2, and at least one memory for storing a program for processing data or the processed data. The server 3 may be, or may include, various computing devices such as a workstation, a cloud, a data drive, a data station, etc. The server 3 may be, or may include, one or more servers physically or logically classified based on function, sub-configuration of the function and/or data, and may transmit and/or receive data through inter-server communication and process the data.

The server 3 may perform functions, such as managing a user account, registering the health care device 1 by connecting the health care device 1 to the user account, and managing or controlling the registered health care device 1. For example, the user may access the server 3 through the user equipment 2 to create a user account. The user account may be identified by an identifier (ID) and a password created by the user. The server 3 may register the health care device 1 with the user account according to a predetermined procedure. The health care device 1 may be a personal device owned by the user, or a public device installed in a fitness center or other health care service company and used by many people. For example, the server 3 may connect identification information (e.g., a serial number, a media access control (MAC) address, etc.) of the health care device 1 to the user account to register, manage and control the health care device 1.

The user equipment 2 may include a communication module for communicating with the health care device 1 and/or the server 3. The user equipment 2 may include a user interface for receiving user inputs or outputting information for the user. The user equipment 2 may include at least one processor for controlling operation of the user equipment 2 and at least one memory for storing a program for controlling the operation of the user equipment 2.

The user equipment 2 may be carried by the user or placed in a home or office of the user. The user equipment 2 may include a personal computer, a terminal, a mobile phone, a smart phone, a handheld device, a wearable device, a display device, etc., without being limited thereto.

In the memory of the user equipment 2, a program for controlling the health care device 1 (e.g., an application) may be stored. The application may be sold in a state of being installed in the user equipment 2, or may be downloaded and installed from an external server.

The user may access the server 3 and create a user account by running the application installed in the user equipment 2, and register the health care device in the server 3 by communicating with the server 3 based on the login user account.

For example, when the health care device 1 is operated to access the server 3 according to a procedure guided in the application installed in the user equipment 2, the server 3 may register the health care device 1 with the user account by registering the identification information (e.g., a serial number or a MAC address) of the health care device 1 with the user account.

The user may use the application installed in the user equipment 2 to control the health care device 1. For example, when the user signs up with the user account in the application installed in the user equipment 2, the health care device 1 registered with the user account may be output through a user interface of the user equipment 2, for example by being display on a display of the user equipment 2. When a control command for the health care device 1 is input to the user equipment 2, the user equipment 2 may forward the control command to the health care device 1 through the server 3.

A network may include both a wired network and a wireless network. The wired network may include at least one of a cable network and a telephone network, and the wireless network may include any network that transmits or receives signals in radio waves. The wired network and the wireless network may be connected to each other.

The network may include a wide area network (WAN) such as the Internet, a local area network (LAN) formed around an access point (AP), and a short-range wireless network without an AP. The short-range wireless network may include BLUETOOTH (IEEE 802.15.1), ZIGBEE (IEEE 802.15.4), wireless fidelity (WI-FI) direct, near field communication (NFC), Z-WAVE, etc., without being limited thereto.

The AP may connect the health care device 1 and/or the user equipment 2 to the WAN connected to the server 3. The health care device 1 and the user equipment 2 may be connected to the server 3 through the WAN.

The AP may use wireless communication such as Wi-Fi (IEEE 802.11), BLUETOOTH (IEEE 802.15.1), ZIGBEE (IEEE 802.15.4), etc., to communicate with the health care device 1 and/or the user equipment 2, and use wired communication to access the WAN, but the wireless communication scheme of the AP is not limited thereto.

In one or more embodiments, the health care device 1 may be directly connected to the user equipment 2 and/or the server 3 without passing through the AP.

The health care device 1 may be connected to the user equipment 2 and/or the server 3 over a long-range wireless network or a short-range wireless network.

For example, the health care device 1 may be connected to the user equipment 2 over a short-range wireless network (e.g., WI-FI, direct or BLUETOOTH). In another example, the health care device 1 may use the long-range wireless network (e.g., a cellular communication module) to connect to the user equipment 2 and/or the server 3 through the WAN.

The health care device 1 may transmit information about an operation or status to the user equipment 2 and/or the server 3 over the network. For example, based on receiving a request from the server 3 or when a particular event occurs in the health care device 1, the health care device 1 may transmit the information about the operation or the status to the user equipment 2 and/or the server 3 periodically or in real time.

Based on receiving the information about the operation or status from the health care device 1, the server 3 may update information about an operation or status of the health care device 1 that has been stored therein, and transmit the updated information about the operation and/or status of the health care device 1 to the user equipment 2 over the network. The updating of the information may include various operations to change the existing information such as adding new information to the existing information, replacing the existing information with new information, etc.

The health care device 1 may obtain various pieces of information from the user equipment 2 and/or the server 3, and provide the obtained information to the user. For example, the health care device 1 may obtain information relating to a function of the health care device 1 or various environmental information (e.g., weather, temperature, humidity, etc.) from the server 3 and output the obtained information through the user interface.

The health care device 1 may operate according to a control command received from the user equipment 2 and/or the server 3. For example, when the health care device 1 has obtained prior approval of the user to operate according to a control command of the server 3 even without having a user input, the health care device 1 may operate according to the control command received from the server 3. The control command received from the server 3 may include a control command input by the user through the user equipment 2, a control command based on a preset condition, or the like, without being limited thereto.

In various embodiments, the health care device 1 may operate according to a control command received from the user equipment 2 even without having a user input. The control command received from the health care device 1 and/or the user equipment 2 may include a control command input by the user through the health care device 1 and/or the user equipment 2, a control command based on a preset condition, or the like, without being limited thereto.

The user equipment 2 may transmit information about the user to the health care device 1 and/or the server 3 through the communication module. For example, the user equipment 2 may transmit information about a location of the user, a physical condition of the user, a preference of the user, a schedule of the user, etc., to the server 3. The user equipment 2 may transmit the information about the user to the server 3 according to prior approval of the user.

The health care device 1, the user equipment 2 and/or the server 3 may determine a control command using an artificial intelligence (AI) technology, such as an AI algorithm or an AI model. For example, the server 3 may process the information about the operation or status of the health care device 1 and the information about the user of the user equipment 2 using the AI technology, and transmit a result of the processing or a control command to the health care device 1 and/or the user equipment 2 based on the result of the processing.

In embodiments, the health care device 1 and/or the user equipment 2 may collect biometric information associated with the user and transmit the information to the server 3 through the communication module. In embodiments, the biometric information associated with the user may be biometric obtained from the user, or biometric information otherwise indicating the user. For example, the biometric information may be or may include information about a body of the user.

The server 3 may receive biometric information associated with each user provided from the health care device 1 and/or the user equipment and build a user database in a database module. The biometric information associated with each user may be registered in the user database.

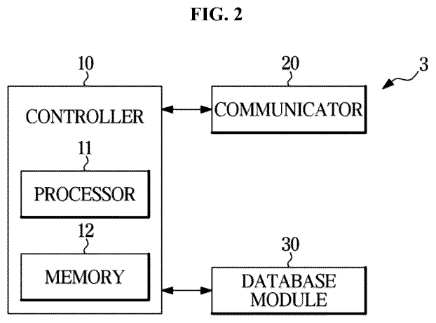

FIG. 2 illustrates an example of a control block of a server, according to an embodiment.

Referring to FIG. 2, the server 3 may include a controller 10, a communicator 20 and a database module 30.

The controller 10 may be electrically connected to the communicator 20 and the database module 30.

The communicator 20 may perform connection to at least one of the health care device 1, the user equipment 2 and the server 3 over a network. The controller 10 may obtain various information, various signals and/or various data from health care device 1, the user equipment 2 and the server 3 through the communicator 20. The controller 10 may transmit various information, various signals and/or various data to the health care device 1, the user equipment 2 and the server 3 through the communicator 20.

The communicator 20 may include various communication circuits. The communicator 20 may include a wireless communication circuit and/or a wired communication circuit. The wireless communication circuit may support various wireless communication such as a wireless LAN, home radio frequency (RF), infrared communication, ultra-wide band (UWB) communication, WI-FI, BLUETOOTH, ZIGBEE and/or a long-range wireless network (e.g., cellular communication).

The controller 10 may control the components of the server 3. The controller 10 may include a processor 11 and a memory 12. The processor 11 may include logic circuits and operation circuits in hardware. The processor 11 may control the electrically connected components of the server 3 based on a program, instructions and/or data stored in the memory 12 for an operation of the server 3. The controller 10 may be implemented with a control circuit including circuit elements such as a condenser, an inductor and a resistor. The processor 11 and the memory 12 may be implemented in separate chips or in a single chip. Furthermore, the controller 10 may include a plurality of processors and a plurality of memories.

The memory 12 may store the program, application and/or data for operation of the server 3 and store data created by the processor 11. The memory 12 may include a non-volatile memory such as a read-only memory (ROM) and a flash memory for long-term storage of the data. The memory 12 may include a volatile memory for short-term or temporary storage of the data, such as a static random-access memory (SRAM) and a dynamic random-access memory (DRAM).

The controller 10 may receive biometric information associated with a user who uses the health care device 1, through the communicator 20. The controller 10 may identify the user based on the received biometric information. The controller 10 may transmit, to the health care device 1, a control command for the health care device 1 to perform a personalized care operation corresponding to the identified user.

The controller 10 may manage a user account of the user who uses the health care device 1. The controller 10 may perform functions, such as registering the health care device 1 by connecting the health care device 1 to the user account, and managing or controlling the registered health care device 1. The controller 10 may register the health care device 1 and/or the user equipment 2 with the user account according to a set procedure. For example, the controller 10 may connect identification information (e.g., a serial number, a media access control (MAC) address, etc.) of the health care device 1 to the user account to register, manage and control the health care device 1.

The database module 30 may store a user database (DB). In the user DB, biometric information associated with each user account of a plurality of users who use the health care device 1 may be registered. The user DB may include a user account DB and a biometric information DB. The user account DB and the biometric information DB may store information associated with each other.

The controller 10 may transmit, to the health care device 1, a control command instructing the health care device 1 to operate according to a mode preferred by the identified user from among control modes of the health care device 1.

The controller 10 may determine a change in condition of the identified user by comparing a current value of the biometric information associated with the identified user with a previous value. The controller 10 may transmit, to the health care device 1, a control command instructing the health care device 1 to perform a personalized care operation corresponding to the change in condition of the identified user.

The controller 10 may transmit, to the health care device 1, a control command instructing the health care device 1 to output or display information about at least one of a recommended mode, medical treatment and a health care method corresponding to the change in condition of the identified user.

The controller 10 may determine a customized mode for the health care device 1 based on the change in condition of the identified user. The controller 10 may transmit, to the health care device 1, a control command for the health care device 1 to operate according to the customized mode. In this case, the customized mode may be at least one of the control modes of the health care device 1 or at least one of the control modes, which has one or more changed mode variables.

The health care device 1 may include the smart bed 1a, the smart massage device 1b and the smart workout equipment 1c.

In a case in which the health care device 1 includes the smart bed 1a, the controller 10 may determine the customized mode to be at least one of control modes of the smart bed 1a based on the change in condition of the identified user, or to be at least one mode of the control modes, in which one or more of bed posture, mattress hardness, heat, humidity, light conditions, etc., have changed.

In a case in which the health care device 1 includes the smart massage device 1b, the controller 10 may determine the customized mode to be at least one of control modes of the smart massage device 1b based on the change in condition of the identified user, or to be at least one mode of the control modes, in which one or more of massage type, massage portion and intensity, heating portion and heating intensity, etc., have changed.

In a case in which the health care device 1 includes the smart workout equipment 1c, the controller 10 may determine the customized mode to be at least one of control modes of the smart workout equipment 1c based on the change in condition of the identified user, or to be at least one mode of the control modes, in which one or more of workout type, workout intensity, etc., have changed.

Accordingly, the server 3 may automatically provide a health care service personalized for each user.

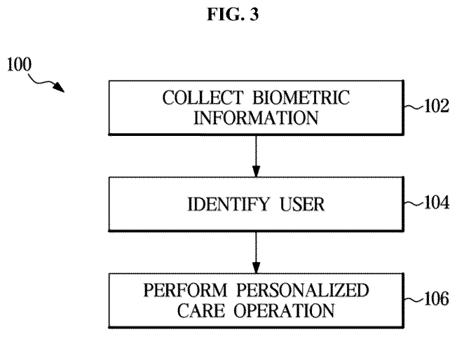

FIG. 3 is a flowchart of a process 100 for describing a method of providing a health care service, according to an embodiment.

Referring to FIG. 3, the health care device 1 may collect biometric information associated with a user who uses the health care device 1 through at least one sensor at operation 102.

The health care device 1 may use the at least one sensor arranged to collect the biometric information associated with the user to collect the biometric information associated with the user who uses the health care device 1. The at least one sensor may collect biometric information, including information about at least one of a height, a weight, a blood pressure, a heart rate, a heart rate variability, an oxygen saturation, a respiratory rate, a body temperature, fingerprints, an iris, a face, a vein, a voice, etc.

The user may be identified based on the user database stored in the server 3 and the biometric information at operation 104. For example, the server 3 may identify the user by comparing the user database stored in the database module 30 of the server 3 with the biometric information. The server 3 may identify the user by comparing the biometric information with biometric information registered in the user database.

The server 3 may identify the user using one or both of physical trait information, such as information about a height and a weight, and the biometric feature information such as information about a blood pressure, a heart rate, a heart rate variability, an oxygen saturation, a respiratory rate, a body temperature, fingerprints, an iris, a face, a vein, a voice, etc. The server 3 may identify the user, and may determine whether the user is a registered user or a new user.

The health care device 1 may perform a personalized care operation corresponding to the identified user at operation 106. The health care device 1 may perform the personalized care operation to provide a health care service optimized for the identified user.

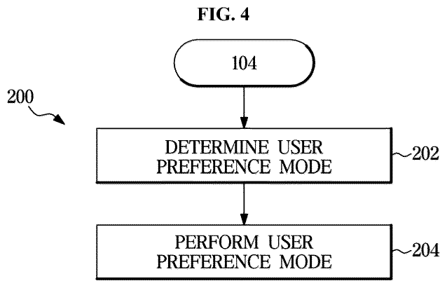

FIG. 4 is a flowchart for describing a process for operating according to a mode preferred by an identified user in a method of providing a health care service, according to an embodiment. In one or more embodiments, the process 200 illustrated in FIG. 4 may be performed after operation 104 discussed above, but embodiments are not limited thereto.

Referring to FIG. 4, according to the process 200, the health care device 1 may determine a user preference mode, which may be mode preferred by the identified user from among control modes of the health care device 1, at operation 202. The user preference mode may be a mode that has a highest use frequency among the control modes of the health care device 1, or a mode having a high use frequency recently, or during a predetermined period. The user preference mode may be a mode that has been most recently used by the user.

The health care device 1 may operate according to the determined user preference mode, at operation 204.

As such, the health care device 1 may perform the personalized care operation corresponding to the identified user by operating according to a mode preferred by the identified user.

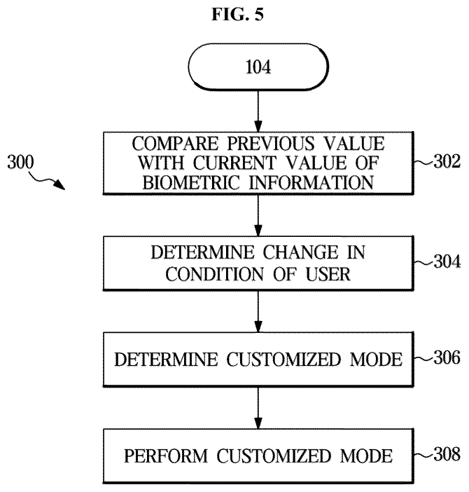

FIG. 5 is a flowchart for describing a process for operating according to a customized mode corresponding to a change in condition of an identified user in a method of providing a health care service, according to an embodiment. In one or more embodiments, the process 300 illustrated in FIG. 5 may be performed after operation 104 discussed above, but embodiments are not limited thereto.

Referring to FIG. 5, according to the process 300, current and previous values of biometric information associated with the identified user may be compared at operation 302. The health care device 1 or the server 3 may compare the current and previous values of the biometric information associated with the identified user.

Based on a result of the comparing between the current and previous values of the biometric information associated with the identified user, a change in condition of the user may be determined at operation 304. The health care device 1 or the server 3 may determine a change in the condition of the user according to the result of the comparing between the current and previous values of the biometric information associated with the identified user. Accordingly, a personalized care operation corresponding to the change in the condition of the identified user may be performed based on the change in condition of the user.

A customized mode for the health care device 1 may be determined based on the change in condition of the identified user at operation 306. The health care device 1 or the server 3 may determine the custom mode to be a control mode of the health care device 1 corresponding to the change in the condition of the identified user. In this case, a customized mode according to a change in condition of each user may be set in advance.

The customized mode may be at least one of the control modes of the health care device 1. The customized mode may be one of the control modes of the health care device 1. The customized mode may be two or more of the control modes of the health care device 1. For example, in a case in which there are 5 control modes, the customized mode may be, or may include, a combination of two modes for example a combination of a first mode and a fourth mode, or a combination of three modes, for example a combination of a third mode, a fourth mode, and a first mode. When the customized mode is a combination of two or more modes, the respective modes may be arranged in a time series.

Furthermore, the customized mode may be a modified mode, which may be, or may include, at least one control mode from among the control modes of the health care device 1 having one or more changed mode variables. For example, when the customized mode is a modified first mode, one or more of a plurality of mode variables of the first mode may be changed. When the customized mode is a modified combination of the first mode and the fourth mode, one or more of a plurality of mode variables of the first mode and/or the fourth mode may have been changed.

For example, when the health care device 1 includes the smart bed 1a, the customized mode may be at least one mode corresponding to a change in condition of the identified user from among control modes of the smart bed 1a. Furthermore, the customized mode may be at least one of the control modes of the smart bed 1a, in which one or more of bed posture, mattress hardness, heat, humidity, and illumination have been changed.

When the health care device 1 includes the smart massage device 1b, the customized mode may be at least one mode corresponding to a change in condition of the identified user from among control modes of the smart massage device 1b. Furthermore, the customized mode may be at least one of the control modes of the smart massage device 1b, in which one or more of massage type, massage portion and intensity, and heating portion and heating intensity have been changed.

When the health care device 1 includes the smart workout equipment 1c, the customized mode may be at least one mode corresponding to a change in condition of the identified user from among control modes of the smart workout equipment 1c. Furthermore, the customized mode may be at least one of the control modes of the smart workout equipment 1c, in which one or more of workout type and workout intensity have been changed.

The health care device 1 may operate according to the determined customized mode at operation 308.

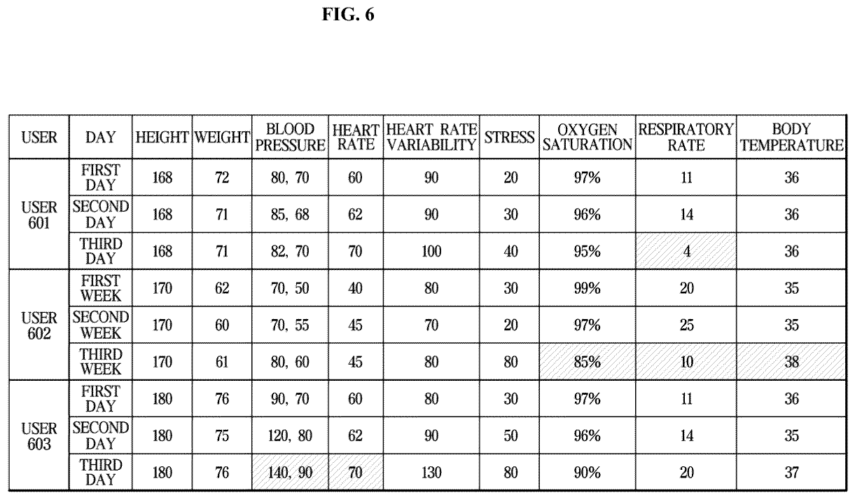

FIG. 6 illustrates a user database stored in a server, according to an embodiment.

Referring to FIG. 6, the user database may store information such as information about a height, a weight, a blood pressure, a heart rate, a heart rate variability, stress, an oxygen saturation and a respiratory rate.

The users may be identified as user 601, user 602 and user 603 based on user accounts.

For example, as shown in FIG. 6, user 601 may be shown to have a height of 168 centimeters, a weight of 71 to 72 kilograms, a heart rate of 60 to 70 beats per minute, a heart rate variability of 90 to 100, an oxygen saturation of 95% to 97%, a respiratory rate of 4 to 11 breaths per minute, and a body temperature of 36° C.

When the identified user corresponds to user 601, a current value of the biometric information associated with user 601 may be compared with the previous value registered in the user database, and based on the result of the comparing, a change in user condition may be determined. The previous value may be an average value of biometric information collected over a predetermined period of time. When a difference between the current and previous values of the biometric information associated with user 601 is outside of a predetermined range, a current condition and a change in condition of user 601 may be determined based on a combination of biometric information outside of the predetermined range. Accordingly, the health care device 1 may perform a personalized care operation corresponding to the change in condition of user 601, so a personalized health care service optimized for the change in user condition may be automatically provided for each user.

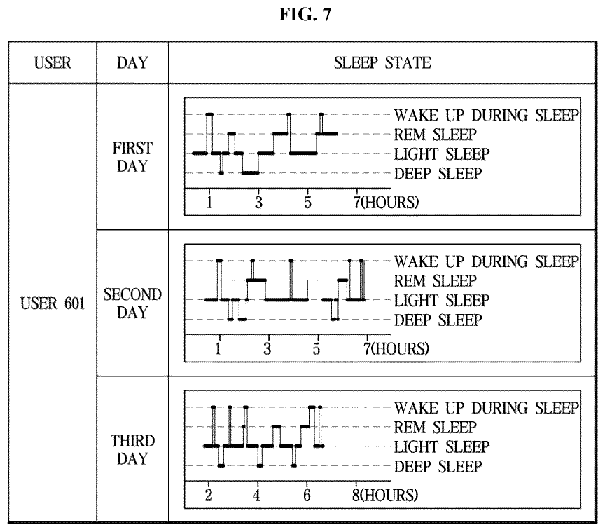

FIG. 7 illustrates a sleep state of each user in a user database stored in a server, according to an embodiment.

Referring to FIG. 7, in the case of user 601, it is shown that deep sleep hours are reduced and waking hours increase from the first day toward the third day.

As such, a change in user's sleep state may be determined by comparing a sleep state value of the biometric information associated with user 601 with a previous sleep state value registered in the user database. Accordingly, the health care device 1 may perform a personalized care operation corresponding to the change in the sleep state of user 601, so a personalized health care service optimized for a change in the sleep state of the user may be automatically provided for each user.

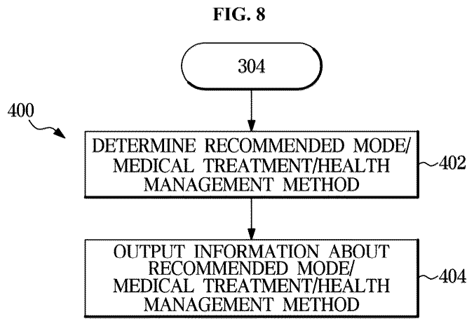

FIG. 8 illustrates a process for outputting information about a recommended mode, medical treatment or a health care method corresponding to a change in a user condition in a method of providing a health care service, according to an embodiment. In one or more embodiments, the process 400 illustrated in FIG. 8 may be performed after operation 304 discussed above, but embodiments are not limited thereto.

Referring to FIG. 8, according to process 400, at least one of a recommended mode, medical treatment and a health care method corresponding to the identified user may be determined based on a change in condition of the identified user, at operation 402. The health care device 1 or the server 3 may determine at least one of the recommended mode, medical treatment and health care method corresponding to the identified user based on a change in condition of the identified user.

The health care device 1 may output information about at least one of the recommended mode, medical treatment and health care method corresponding to the identified user, at operation 404.

Referring to FIGS. 6 and 7, in the case of user 601, the user may wakeup during sleep more frequently on the third day than on the first day and the second day, and the respiratory rate drops to 4 on the third day as compared to 11 on the first day and 14 on the second day, which indicates that sleep apnea occurs. Accordingly, the health care device 1 may give user 601 a simple stimulus in response to the change in user condition, and may suggest fluid intake before sleeping and proper sleeping hours by analyzing a previous sleep pattern that shows the most reliable sleeping experience.

In a case of user 602, the user condition may be abnormal because the oxygen saturation is reduced to 85% from 97%, the respiratory rate is reduced to 10 from 25, the body temperature rises to 38° C. from 35° C. in the third week, as compared to the second week. Accordingly, with consideration for this change in user condition, the health care device 1 may not operate according to any mode of the health care device 1 but may suggest that user 602 go to see a doctor.

In a case of user 603, the health care device 1 may perform, for example, a stretching mode on the second day. As for user 603, although the user condition was not abnormal until the second day, the user condition may become abnormal on the third day with a relatively significant increase in blood pressure of user 603 to 140/90 from a blood pressure of 120/80 on the previous day and a relatively significant increase in heart rate to 70 from 62. Accordingly, the health care device 1 may suggest and then automatically operate according to a meditation mode, which is a recommended mode, instead of the stretching mode that was performed on the second day, by considering the change in user condition.

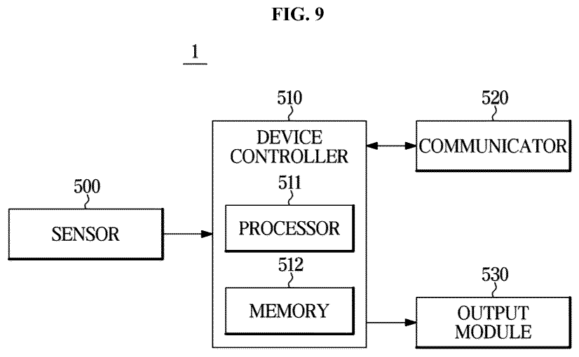

FIG. 9 illustrates an example of a control block of a health care device, according to an embodiment.

Referring to FIG. 9, the health care device 1 according to an embodiment may include at least one sensor 500, a device controller 510, a communicator 520 and an output module 530.

The device controller 510 may be electrically connected to the at least one sensor 500, the communicator 520 and the output module 530.

The at least one sensor 500 may collect biometric information associated with a user who uses the health care device 1.

The at least one sensor 500 may include at least one of a camera, a fingerprint sensor, an eye sensor, a face sensor, a voice sensor, a weight sensor, a blood pressure sensor, a piezo-electric sensor, a heart rate sensor, a respiratory sensor, a body temperature sensor, or a blood amount sensor. It is not, however, limited thereto, and there may be more various sensors capable of collecting biometric information associated with the user. The at least one sensor 500 may use one sensor or a sensor combination to collect information about a height, a weight, a blood pressure, a heart rate, a heart rate variability, an oxygen saturation, a respiratory rate, a body temperature, fingerprints, an iris, a face, a vein, a voice, a snoring sound, etc.

A type of the at least one sensor 500 may vary based on the type of the health care device 1.

The device controller 510 may control the components of the health care device 1. The device controller 510 may include a processor 511 and a memory 512. The processor 511 may include logic circuits and operation circuits in hardware. The processor 511 may control the components of the health care device 1 based on a program, instructions and/or data stored in the memory 512 for an operation of the health care device 1. The controller 510 may be implemented using a control circuit including circuit elements such as a condenser, an inductor and a resistor. The processor 511 and the memory 512 may be implemented in separate chips or in a single chip. Furthermore, the device controller 510 may include a plurality of processors and a plurality of memories.

The memory 512 may store the program, application and/or data for the operation of the health care device 1, and store data created by the processor 511. The memory 512 may include a non-volatile memory such as a ROM and a flash memory for long-term storage of the data for a long time. The memory 512 may include a volatile memory for short-term or temporary storage of the data, such as an SRAM and a DRAM.

The communicator 520 may connect to at least one of another health care device 1, the user equipment 2 and the server 3 over a network. The device controller 510 may obtain various information, various signals and/or various data from the other health care device 1, the user equipment 2 and the server 3 through the communicator 520. The device controller 510 may transmit the various information, various signals and/or various data to the other health care device 1, the user equipment 2 and the server 3 through the communicator 520.

The communicator 520 may include various communication circuits. The communicator 520 may include a wireless communication circuit and/or a wired communication circuit. The wireless communication circuit may support various wireless communication such as a wireless LAN, home RF, infrared communication, UWB communication, WI-FI, BLUETOOTH, ZIGBEE and/or a long-range wireless network (e.g., cellular communication).

The output module 530 may include components related to an output of the health care device 1.

The output module 530 may include a display (e.g., a liquid crystal display (LCD) panel, a light emitting diode (LED) panel, a light emitting diode (LED) module and/or a speaker.

For example, when the health care device 1 includes the smart bed 1a, the output module 530 may include a bed posture control device, a mattress hardness control device, a heating device, a humidity device, an illumination device, etc., in addition to the display and/or the speaker.

Furthermore, when the health care device 1 includes the smart massage device 1b, the output module 530 may include a massage device, a heating device, etc., in addition to the display and/or the speaker.

Furthermore, when the health care device 1 includes the smart workout equipment 1c, the output module 530 may include a workout intensity control device in addition to the display and/or the speaker.

The device controller 510 may transmit information collected through the at least one sensor 500 to the server 3 through the communicator 520.

The device controller 510 may receive identity information of the user from the server 3 through the communicator 520.

The device controller 510 may control the output module 530 to perform a personalized care operation corresponding to the user identified by the identification information of the user.

The device controller 510 may receive information about a change in condition of the identified user from the server 3 through the communicator 520.

The device controller 510 may control the output module 530 to perform a personalized care operation corresponding to the change in condition of the identified user.

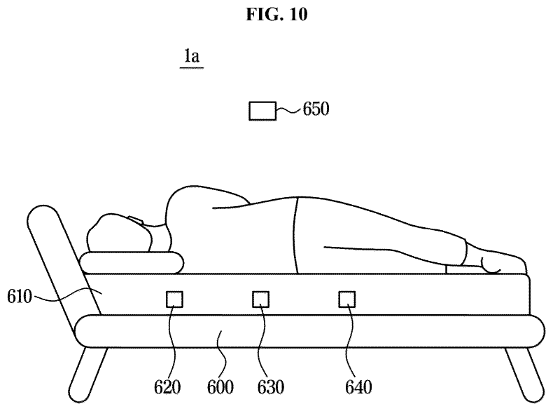

FIG. 10 illustrates an example in which a health care device includes a smart bed, according to an embodiment.

Referring to FIG. 10, the smart bed 1a may include a bed frame 600 and a smart mattress 610.

The user may lie on the smart mattress 610 arranged on the bed frame 600.

The smart bed 1a may include a bed posture control device 620 for controlling bed posture by changing the shape of the smart mattress 610 and controlling hardness of the smart mattress 610, a heating device 630 for providing heat to the smart mattress 610, a humidity device 640 for controlling humidity of the smart mattress 610, an illumination device 650 for controlling illumination around the smart bed 1a, etc. In embodiments, at least one of the bed posture control device 620, the heating device 630, and the illumination device 650 may be included in an output module (e.g., the output module 530) in addition to the display and/or the speaker. However, embodiments are not limited thereto, and the smart bed 1a may further include various devices that may bring a change in operation of the smart bed 1a.

The smart bed 1a may use the at least one sensor 500 to collect biometric information associated with the user who lies on the smart mattress 610, such as information about a height, a weight, a blood pressure, a heart rate, a heart rate variability, an oxygen saturation, a respiratory rate, a body temperature, a fingerprint, an iris, a face, a vein, a voice, a snoring sound, a sleeping condition, etc.

The smart bed 1a may operate according to a user preference mode corresponding to the identified user from among the control modes of the smart bed 1a. The smart bed 1a may perform a personalized care operation corresponding to the identified user by performing the user preference mode.

The smart bed 1a may operate according to a customized mode corresponding to a change in condition of the identified user.

The smart bed 1a may operate according to at least one mode corresponding to a change in condition of the identified user among the control modes of the smart bed 1a as the customized mode.

The smart bed 1a may operate according to at least one of the control modes, in which one or more of bed posture, mattress hardness, heat, humidity, and illumination have been changed, as the customized mode.

Accordingly, the smart bed 1a may perform a personalized care operation to provide a sleep care service optimized for the identified user.

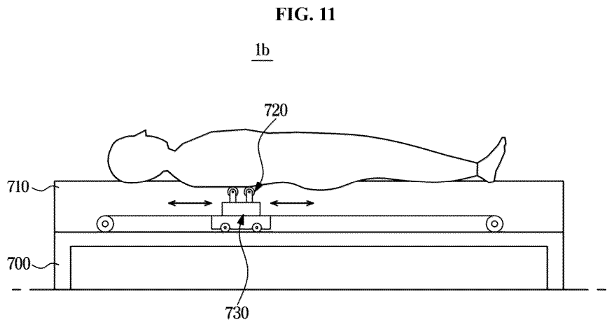

FIG. 11 illustrates an example in which a health care device includes a smart massage device, according to an embodiment.

Referring to FIG. 11, the smart massage device 1b may include a frame 700 and a mat 710.

The user may lie on the mat 710 arranged on the frame 700.

The smart massage device 1b may include a massage device 720 that is movable within the mat 710 for massaging the user's body portions and a heating device 730 for providing heat to the user's body portions, etc. In embodiments, at least one of the massage device 720 and the heating device 730 may be included in an output module (e.g., the output module 530) in addition to the display and/or the speaker. However, embodiments are not limited thereto, and the output module may further include various devices that may bring a change in operation of the smart massage device 1b.

The smart massage device 1b may use the at least one sensor 500 to collect biometric information associated with the user who uses the smart massage device 1b, such as information about a height, a weight, a blood pressure, a heart rate, a heart rate variability, an oxygen saturation, a respiratory rate, a body temperature, a fingerprint, an iris, a face, a vein, a voice, etc.

The smart massage device 1b may operate according to a user preference mode corresponding to the identified user from among the control modes of the smart massage device 1b. The smart massage device 1b may perform a personalized care operation corresponding to the identified user by performing the user preference mode.

The smart massage device 1b may operate according to a customized mode corresponding to a change in condition of the identified user.

The smart massage device 1b may operate according to at least one mode corresponding to a change in condition of the identified user among the control modes of the smart massage device 1b as the customized mode.

The smart massage device 1b may operate according to at least one of the control modes, in which one or more of massage type, massage portion and intensity, and heating portion and heating intensity have been changed, as the customized mode.

Accordingly, the smart massage device 1b may perform a personalized care operation to provide a massage care service optimized for the identified user.

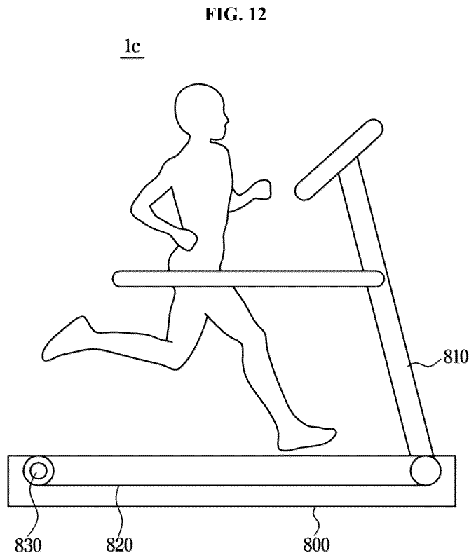

FIG. 12 illustrates an example in which a health care device includes smart workout equipment, according to an embodiment.

Referring to FIG. 12, the smart workout equipment 1c may include a lower frame 800, a support frame 810 and a running belt 820 arranged on the lower frame 800.

The user may run on the running belt 820 arranged on the lower frame 800.

The smart workout equipment 1c may include a workout intensity control device 830 for controlling the running belt 820 to adjust the workout intensity. In embodiments, the workout intensity control device 830 may be included in an output module (e.g., the output module 530) in addition to the display and/or speaker. However, embodiments are not limited thereto, and the output module may further include various devices that may bring a change in operation of the smart workout equipment 1c.

The smart workout equipment 1c may use the at least one sensor 500 to collect biometric information associated with the user who uses the smart workout equipment 1c, such as information about a height, a weight, a blood pressure, a heart rate, a heart rate variability, an oxygen saturation, a respiratory rate, a body temperature, a fingerprint, an iris, a face, a vein, a voice, etc.

The smart workout equipment 1c may operate according to a user preference mode corresponding to the identified user among the control modes of the smart workout equipment 1c. The smart workout equipment 1c may perform a personalized care operation corresponding to the identified user by performing the user preference mode.

The smart workout equipment 1c may operate according to a customized mode corresponding to a change in condition of the identified user.

The smart workout equipment 1c may operate according to at least one mode corresponding to a change in condition of the identified user among the control modes of the smart workout equipment 1c as the customized mode.

The smart workout equipment 1c may operate according to at least one of the control modes of the smart workout equipment 1c, in which one or more of exercise type exercise intensity have been changed, as the customized mode.

Accordingly, the smart workout equipment 1c may perform a personalized care operation to provide a workout care service optimized for the identified user.

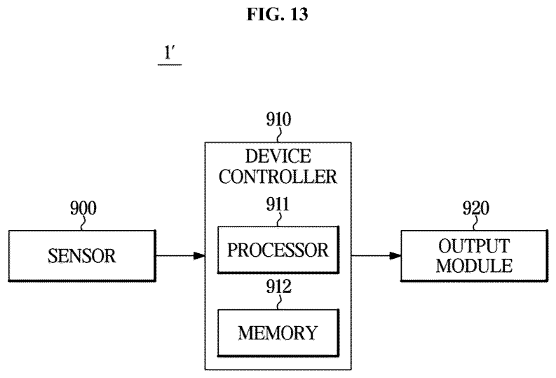

FIG. 13 illustrates an example of a control block of a health care device, according to another embodiment.

Referring to FIG. 13, a health care device 1′ may include at least one sensor 900, a device controller 910, and an output module 920.

The device controller 910 may be electrically connected to the at least one sensor 900 and the output module 920.

The at least one sensor 900 may collect biometric information associated with a user who uses the health care device 1′.

The device controller 910 may identify a user based on the biometric information collected through the at least one sensor 900 and a user database stored in the memory 912. For example, the device controller 910 may identify the user by comparing the biometric information with biometric information registered in the user database.

The device controller 910 may identify the user by using one or both physical trait information such as height and weight and the biometric feature information such as information about a blood pressure, a heart rate, a heart rate variability, an oxygen saturation, a respiratory rate, a body temperature, fingerprints, an iris, a face, a vein, a voice, etc. The device controller 910 may determine whether the user is a registered user or a new user.

The device controller 910 may control the output module 920 to perform a personalized care operation corresponding to the identified user.

The device controller 910 may determine a change in condition of the user according to a result of the comparison between the current value of the biometric information and previous values of the biometric information associated with the identified user.

Hence, the health care device 1′ may perform a personalized care operation to provide a health care service optimized for the identified user.

According to the disclosure, a user may be identified and a health care service optimized and personalized for the identified user may be provided.

According to an embodiment, a method of providing a health care service may include collecting, by the health care device 1, biometric information associated with a user who uses the health care device, through the at least one sensor 500; identifying the user based on the biometric information and a user database stored in a server 3; and performing, by the health care device, a personalized care operation corresponding to the identified user.

The identifying of the user may include identifying the user by comparing the biometric information with biometric information registered in the user database.

The performing of the personalized care operation may include performing a mode preferred by the identified user from among control modes of the health care device.

The method may further include determining a change in condition of the identified user by comparing a current value of the biometric information associated with the identified user with a previous value, and the performing of the personalized care operation may include performing the personalized care operation based on the change in condition of the identified user.

The biometric information may include at least one of information about a height, a weight, a blood pressure, a heart rate, a heart rate variability, an oxygen saturation, a respiratory rate, a body temperature, fingerprints, an iris, a face, a vein, a voice, and a snoring sound.

The previous value may be an average value of biometric information obtained over a predetermined period of time.

The performing of the personalized care operation may further include showing at least one of a recommended mode, medical treatment and a health care method to the identified user based on the change in condition of the identified user.

The method may further include determining a customized mode for the health care device based on the change in the condition of the identified user, and the performing of the personalized care operation may include performing the customized mode.

The customized mode may be at least one control mode from among the control modes of the health care device, or at least one modified mode from among the control modes, having one or more changed mode variables.

Based on the health care device including the smart bed 1a, the customized mode may be at least one mode corresponding to the change in condition of the identified user from among control modes of the smart bed or at least one of the control modes, in which one or more of bed posture, mattress hardness, heat, humidity and illumination have been changed.

Based on the health care device including the smart massage device 1b, the customized mode may be at least one mode corresponding to the change in condition of the identified user from among control modes of the smart massage device or at least one of the control modes, in which one or more of massage type, massage portion and intensity, or heating portion and heating intensity have been changed.

Based on the health care device including the smart workout equipment, the customized mode may be at least one mode corresponding to the change in condition of the identified user from among control modes of the smart workout equipment or at least one of the control modes, in which one or more of workout type and workout intensity have been changed.

According to an embodiment, a server may include a communicator 20; and a controller 10 for receiving biometric information associated with a user who uses a health care device, through the communicator 20, identifying the user based on the biometric information, and transmitting, to the health care device, a control command for the health care device to perform a personalized care operation corresponding to the identified user.

The controller may transmit, to the health care device, a control command to operate according to a mode preferred by an identified user from among control modes of the health care device.

The controller may determine a change in condition of the identified user by comparing a current value of the biometric information associated with the identified user with a previous value, and transmit, to the health care device, a control command to perform a personalized care operation corresponding to the change in condition of the identified user.

The controller may transmit, to the health care device, a control command to show at least one of a recommended mode, medical treatment and a health care method corresponding to the change in condition of the identified user.

The controller may determine a customized mode for the health care device based on the change in condition of the identified user, and transmit, to the health care device, a control command for the health care device to operate according to the customized mode.

The customized mode may be at least one of the control modes of the health care device or at least one of the control modes, which has one or more changed mode variables.

According to an embodiment, a health care device may include the output module 530; the communicator 520; at least one sensor 500 for collecting biometric information associated with a user; and the device controller 510 for transmitting information collected through the at least one sensor to a server through the communicator, receiving user identity information from the server, and controlling the output module to perform a personalized care operation corresponding to the identified user.

The device controller may receive information about a change in condition of the identified user from the server, and control the output module to perform a personalized care operation corresponding to the change in condition of the identified user.

Embodiments of the disclosure may be implemented in the form of a storage medium for storing instructions to be carried out by a computer. The instructions may be stored in the form of program codes, and when executed by a processor, may generate program modules to perform operation in the embodiments of the disclosure.

The machine-readable storage medium may be provided in the form of a non-transitory storage medium. The term ‘non-transitory storage medium’ may mean a tangible device without including a signal, e.g., electromagnetic waves, and may not distinguish between storing data in the storage medium semi-permanently and temporarily. For example, the non-transitory storage medium may include a buffer that temporarily stores data.

In an embodiment of the disclosure, the aforementioned method according to the various embodiments of the disclosure may be provided in a computer program product. The computer program product may be a commercial product that may be traded between a seller and a buyer. The computer program product may be distributed in the form of a storage medium (e.g., a compact disc read only memory (CD-ROM)), through an application store (e.g., play store™), directly between two user devices (e.g., smart phones), or online (e.g., downloaded or uploaded). In the case of the online distribution, at least part of the computer program product (e.g., a downloadable app) may be at least temporarily stored or arbitrarily created in a storage medium that may be readable to a device such as a server of the manufacturer, a server of the application store, or a relay server.

Some embodiments are described above with reference to accompanying drawings. It will be obvious to those of ordinary skill in the art that the disclosure may be practiced in other forms than the embodiments of the disclosure as described above without changing the scope of the disclosure. The above embodiments of the disclosure are only presented as an example, and should not be construed as limiting the present disclosure.

What is claimed is:

1. A method of providing a health care service, the method comprising:
collecting, by a health care device, biometric information associated with a user corresponding to the health care device, through at least one sensor;
identifying the user based on the biometric information and a user database stored in a server; and
performing, by the health care device, a personalized care operation corresponding to the identified user.

2. The method of claim 1, wherein the identifying the user comprises identifying the user by comparing the biometric information with registered biometric information stored in the user database.

3. The method of claim 1, wherein the performing the personalized care operation comprises operating according to a mode that is preferred by the identified user from among a plurality of control modes of the health care device.

4. The method of claim 1, further comprising determining a change in a condition of the identified user by comparing a current value of the biometric information with a previous value of the biometric information,
wherein the performing the personalized care operation comprises performing the personalized care operation based on the change in the condition of the identified user.

5. The method of claim 4, wherein the biometric information comprises information about at least one of a height, a weight, a blood pressure, a heart rate, a heart rate variability, an oxygen saturation, a respiratory rate, a body temperature, fingerprints, an iris, a face, a vein, a voice, or a snoring sound.

6. The method of claim 4, wherein the previous value comprises an average value of the biometric information obtained over a predetermined period of time.

7. The method of claim 4, wherein the performing the personalized care operation comprises displaying information about at least one of a recommended mode, a medical treatment, and a health care method to the identified user based on the change in the condition of the identified user.

8. The method of claim 4, further comprising determining a customized mode for the health care device based on the change in the condition of the identified user,
wherein the performing the personalized care operation comprises operating the health care device according to the customized mode.

9. The method of claim 8, wherein the customized mode comprises at least one mode from among a plurality of control modes of the health care device, or at least one modified mode having one or more changed mode variables from among the plurality of control modes.

10. The method of claim 8, wherein based on the health care device comprising a smart bed, the customized mode comprises at least one control mode corresponding to the change in the condition of the identified user from among a plurality of control modes of the smart bed, or at least one modified mode from among the plurality of control modes in which one or more of a bed posture, a mattress hardness, a heat, a humidity and an illumination is changed.

11. The method of claim 8, wherein based on the health care device comprising a smart massage device, the customized mode comprises at least one control mode corresponding to the change in the condition of the identified user from among a plurality of control modes of the smart massage device, or at least one modified mode from among the plurality of control modes in which one or more of a massage type, a massage portion, a massage intensity, a heating portion, and a heating intensity is changed.

12. The method of claim 8, wherein based on the health care device comprising smart workout equipment, the customized mode comprises at least one control mode corresponding to the change in the condition of the identified user from among a plurality of control modes of the smart workout equipment, or at least one modified mode from among the plurality of control modes in which one or more of a workout type and a workout intensity is changed.

13. A server comprising:
a communicator; and
a controller configured to:
receive biometric information associated with a user who uses a health care device through the communicator,
identify the user based on the biometric information, and
transmit, to the health care device, a control command instructing the health care device to perform a personalized care operation corresponding to the identified user.

14. The server of claim 13, wherein the controller is further configured to transmit, to the health care device, a control command instructing the health care device to operate according to a mode that is preferred by the identified user from among a plurality of control modes of the health care device.

15. The server of claim 13, wherein the controller is further configured to determine a change in a condition of the identified user by comparing a current value of the biometric information with a previous value of the biometric information, and transmit, to the health care device, a control command instructing the health care device to perform a personalized care operation corresponding to the change in the condition of the identified user.
